(19) United States
(12) Patent Application Publication
(10) Pub. No.: US 2012/0115184 A1
(43) Pub. Date: May 10, 2012

(54) METHOD FOR THE DETECTION OF MASTITIS AND MILK QUALITY AND MASTITIS SENSOR

(75) Inventors: Toonika Rinken, Tartu (EE); Raivo Jaaniso, Tartu (EE)

(73) Assignee: University of Tarty, Tartu (EE)

(21) Appl. No.: 13/377,080

(22) PCT Filed: Jun. 9, 2009
(86) PCT No.: PCT/EE09/00009

§ 371 Date: Jan. 23, 2012

(51) Int. Cl. C12Q 1/04; C12M 1/34

(52) U.S. Cl. 435/34; 435/287.1

(57) ABSTRACT

A mastitis sensor and a method for detection of mastitis and determination of milk quality in real time (on-line). Methods and apparatuses for the rapid non-invasive determination of the concentration of dissolved molecular oxygen in milked milk are disclosed. Mastitis sensors are disclosed that include a fiberoptic, amperometric or potentiometric device for the determination of oxygen concentration, a device for data acquisition and processing, mastitis indicator and a device that generates a signal for the automatic on-line elimination of substandard milk of infected animals to prevent the pollution of bigger quantities of milk.

### CROSS-REFERENCE TO RELATED APPLICATIONS

This application is a national phase application pursuant to 35 U.S.C. §371 of International Application No. PCT/EE2009/000009, filed Jun. 9, 2009.

### TECHNICAL FIELD OF THE INVENTION

The present invention relates to the fields of veterinary and milk production, particularly to the determination of raw milk quality. The invention is useful for rapid detection of mastitis and other inflammatory processes in real time, but also for the quick determination of the quality of raw milk and on-line separation of substandard milk to avoid the pollution of dairy production.

### BACKGROUND OF THE INVENTION

Mastitis is the most common infection of dairy cattle and it causes economic losses, being a major problem in the whole world.

The diagnosis of mastitis is currently based on different non-invasive methods:
detection of somatic cell count (SCC) in milk spectrometrically (patent application US2008000426; Grabek et al., 2008) or viscosimetrically, where anionic surfactant is added to the milk, causing the formation of gel of the proteins in somatic cells, which viscosity is measured and calibrated against the somatic cell concentration (U.S. Pat. No. 2,935,384; Schalm, O., Noorlander, D., 1960);
detection of lactate in milk (patent EP1192460, Agresearch, 2003)—bacteria causes the elevated concentration of lactate in the udder (anaerobic environment);
measuring the conductivity of milk; this method is relatively nonspecific, as milk conductivity is influenced by other factors than bacteria and normal biological variation in conductivity has nothing to do with mastitis. (Kamphuis C. Making sense of sensor data: detecting clinical mastitis in automatic milking systems. Dissertation. Faculty of Veterinary Medicine, Utrecht University, the Netherlands, 2010);
monitoring the ratios of various ions in milk. As the level of mastitic infection progresses, the concentration of sodium ions increases and potassium ions decreases (international patent application WO/2006/127921, Westfall, G., 2006);
detection of MAA. In response to an infection, mammalian immune system produces acute phase proteins, e.g. Milk Amyloid A (MAA) protein is produced in cow's udder (“PHASE”™ Milk Amyloid A (MAA) Assay Cat. No.: TP-807, Tridelta Development Limited (Ireland), www.trideltaltd.com);
microbiological tests for the detection of mastitis-causing bacteria, e.g. RAPIDEC Staph tests for the detection of S. aureus (analysis time 24 h) (Boerlin, P. et al., J Clin Microbial., 2003, 41(2):767-771);
spectrophotometric methods based on the application of chemical reagents (patents CN100460866, Ox. Biolog. Tech. Co. Ltd, 2009 and U.S. Pat. No. 6,979,550 Rivas et al., 2005), which produce a coloured product with the detectable compound;
detection method based on infrared thermography (international patent application WO0057164, Emerge Interactive Inc., 2000);
fresh milk is centrifuged in special pipettes and pathogens are detected by the number of cells in different sedimentation layers (Garcia-Cordero, J. L., Barrett L. M., O'Kennedy, R & Ricco, A. J. Microfluidic Sedimentation Cytometer for Milk Quality and Bovine Mastitis Monitoring. Biomedical Microdevices, 12:1051-1059, 2010).
the chemiluminescence assay is used to measure the ability of phagocytes to emit light after bacterial invasion (Takahashi, H. “Cytokine Therapy for Staphylococcus Mastitis in Dairy Cows” Science & Technonews Tsukuba, 1999, 50:55-56).

Another disclosed approach is the method of mastitis detection, which is based on the determination of lactate in milk and comparing the lactate level with the lactate levels of healthy animals' milk (U.S. Pat. No. 7,033,836, Pastoral Agric. Res. Inst. Nz Ltd., 2006).

However, the above methods have several disadvantages:
Relatively low lifetime of the sensor. For example, the lactate sensor needs frequent renewal, as its recognition system is based on enzymes. The (optical) oxygen sensor, applied in the present invention, can be operated for years;
Lactate concentration in milk depends on many different factors—feeding, milking frequency, lactation phase etc.;
Most of the abovementioned methods are not applicable on-line in real time course and it is not possible to eliminate substandard milk in the course of milking;
Some methods require addition of different compounds to the milk;
Some methods require costly equipment and highly-qualified personnel.

In scientific studies, the dissolved oxygen concentration in udder (before milking) has been studied with the purpose of studying whether the dissolved oxygen content in udder of normal cows and those of mastitis were sufficient to support normal neutrophil, function to eliminate S. aureus. Neutrophils kill bacteria by 2 methods: oxidative and non-oxidative. When neutrophils are stimulated to phagocytose, there will be an increase in oxygen consumption and the production of oxygen radicals (e.g., superoxide), resulting from the activation of NADPH oxidase, which forms an electron transport chain converting molecular O₂ to superoxide. It was found, that mastitis led to a dramatic drop in O₂ concentration and the antimicrobial activity of neutrophils in udder was depressed. Normal cows have the levels of dissolved O₂ in milk similar to those in venous blood; the levels of dissolved O₂ in mastitic cows are less than 10% of control values (Mayer S J, Waterman A E, Keen P M, Craven N, Bourne J. “Oxygen concentration in milk of healthy and mastitic cows and implications of low oxygen tension for the killing of Staphylococcus aureus by bovine neutrophils.” Journal of Dairy Research 1988; 55(4): 513-9).

There are no methods known in which the determination of dissolved O₂ in milk have been used for the detection of mastitis.

### BRIEF DESCRIPTION OF THE DRAWINGS

For the present invention to be clearly understood and readily practiced, the present invention will be described in conjunction with the following FIGURES, wherein like reference characters designate the same or similar elements, which figures are incorporated into and constitute a part of the specification, wherein:

FIG. 1 displays a histogram of oxygen concentration data in freshly milked milk (385 measurements) and the approximation of these data to the normal distribution (continuous line). ◯—results close to the mean value (377 measurements); —outliers (8 measurements).

### DISCLOSURE OF THE INVENTION

The present invention proposes a quick and reliable method for the detection of mastitis and the determination of the quality of milk in real time and a mastitis sensor. The method for the detection of mastitis and the determination of the quality of milk is based on the noninvasive measurement of dissolved molecular oxygen in milk. In this instance, the oxygen concentration is substantially bigger or smaller from the normal concentration of dissolved oxygen in milk (difference is more than 3 standard deviation σ values from the mean value of the typical concentration or other given threshold), mastitis or other inflammatory processes in the organism of the animal and the substandard milk are detected on-line. In milking systems, based on the application of vacuum devices, the measurement of oxygen is carried out as quickly as possible, but not later than 60 seconds after the beginning of the milking process to avoid the mass transfer of oxygen from air. The oxygen concentration is measured in milk from one or several teats.

The mastitis sensor comprises a fiberoptic, amperometric or potentiometric device for the determination of oxygen concentration; a device for data acquisition and processing; mastitis indicator and a device, generating a signal for the automatic on-line elimination of substandard milk. The mastitis sensor is used for the application of the method, described in the present invention for the detection of mastitis and the determination of the quality of milk.

Proposed in the present invention methods and mastitis sensor have several advantages in comparison with earlier solutions, as they allow:
to detect quickly the potential mastitis sources in subclinical phase in milking animals, like cows, goat, sheep etc.;
to detect mastitis and other inflammatory processes and determine the quality of milk on-line;
to remove the infected animals' substandard milk before milk collecting tank;
the application of mastitis sensor is very easy and does not require special skills.

Maintenance costs of the proposed method and device are low due to the long lifetime of the sensors and fact, that there is no regular need for waste materials or renewal of the system components. Detection of infected animals in real time enables the reduction of production costs, as the substandard milk can be eliminated early in the milking process and the pollution of bigger amounts of milk prevented. There is no need for time-consuming and expensive analytical procedures. Application of the proposed device does not require special training of the personnel. The detection of animals with sub-clinical mastitis in the early phase of the infection allows starting early treatment of the animals with more effective results. So the application of the method and device gives economic effect in reducing both the steady and running costs of production, but also in the improvement of animal welfare. The following examples illustrate the application of the invention, although the invention is not limited with the following examples, but can be applied according to the claims.

### DESCRIPTION OF EMBODIMENTS

Method for the detection of mastitis and determination of milk quality was used in a farm, where we measured the concentration of dissolved oxygen with a Clark-type sensor in the milk of 385 cows. Milk probes of 12 ml were taken from the milking device and oxygen was measured in the probes as quickly as possible, but not later than 60 seconds after taking the probes from the device to prevent the mass transfer of oxygen from the surrounding air into milk.

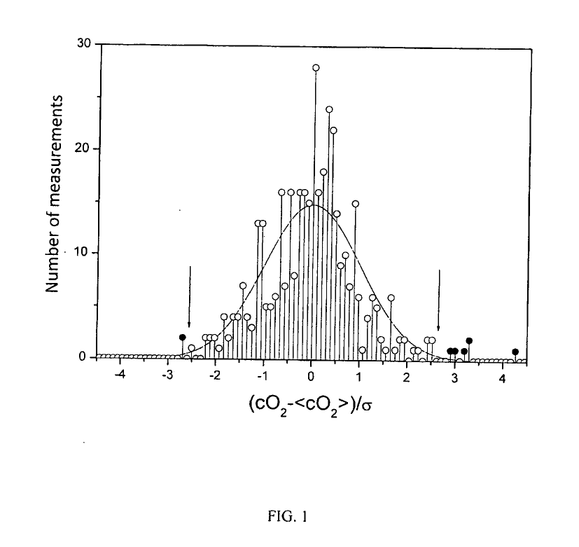

We analyzed the obtained results and calculated the normalized mean value and the standard deviation σ of oxygen concentration. The results are shown on FIG. 1 and Table 1.

TABLE 1
The normalized mean concentration of oxygen in milk of
different cows.

| | cO₂ | | cO₂ | | cO₂ | | cO₂ |
|---|---|---|---|---|---|---|---|
| | normal- | | normal- | | normal- | | normal- |
| No. | ized | No. | ized | No. | ized | No. | ized |
| 1. | 0.8410041 | 98. | 1.018828 | 195. | 1.023013 | 292. | 0.9767441 |
| 2. | 0.7175732 | 99. | 1.108787 | 196. | 0.9853556 | 293. | 1.023256 |
| 3. | 0.8556485 | 100. | 1.087866 | 197. | 1.002092 | 294. | 0.9790697 |
| 4. | 1.010460 | 101. | 1.010460 | 198. | 1.002092 | 295. | 1.006977 |
| 5. | 1.056485 | 102. | 1.104602 | 199. | 1.012552 | 296. | 1.074419 |
| 6. | 0.9707112 | 103. | 1.135983 | 200. | 1.129707 | 297. | 1.065116 |
| 7. | 0.8807531 | 104. | 0.9958159 | 201. | 0.9707112 | 298. | 0.9186046 |
| 8. | 0.9205021 | 105. | 1.129707 | 202. | 1.012552 | 299. | 1.097674 |
| 9. | 0.9748953 | 106. | 1.064854 | 203. | 1.020921 | 300. | 1.134884 |
| 10. | 0.9225941 | 107. | 1.041841 | 204. | 0.916318 | 301. | 0.8930232 |
| 11. | 0.878661 | 108. | 1.008368 | 205. | 0.9016736 | 302. | 1.172093 |
| 12. | 0.9435146 | 109. | 1.043933 | 206. | 0.4651163 | 303. | 1.074419 |
| 13. | 0.962343 | 110. | 0.9958159 | 207. | 1.144186 | 304. | 1.090698 |
| 14. | 0.7740586 | 111. | 1.309623 | 208. | 1.090698 | 305. | 1.044186 |
| 15. | 0.9832635 | 112. | 1.138075 | 209. | 0.8860465 | 306. | 1.569767 |
| 16. | 0.9790794 | 113. | 1.215481 | 210. | 1.046512 | 307. | 1.083721 |
| 17. | 0.9058577 | 114. | 1.284519 | 211. | 0.9186046 | 308. | 1.081395 |
| 18. | 0.9414226 | 115. | 1.012552 | 212. | 0.9186046 | 309. | 1.006977 |
| 19. | 0.9205021 | 116. | 1.152720 | 213. | 1.104651 | 310. | 1.058140 |
| 20. | 0.9832635 | 117. | 1.223849 | 214. | 0.8116279 | 311. | 1.453488 |
| 21. | 1.016736 | 118. | 1.148535 | 215. | 0.7953488 | 312. | 1.034884 |
| 22. | 0.9205021 | 119. | 1.046025 | 216. | 1.046512 | 313. | 1.093023 |
| 23. | 0.962343 | 120. | 1.096234 | 217. | 0.9186046 | 314. | 1.109302 |
| 24. | 0.9518828 | 121. | 1.037657 | 218. | 0.9813952 | 315. | 1.067442 |
| 25. | 0.9351463 | 122. | 1.085774 | 219. | 0.9604651 | 316. | 1.141860 |
| 26. | 0.8849372 | 123. | 1.046025 | 220. | 1.093023 | 317. | 1.044186 |
| 27. | 0.8033472 | 124. | 1.014644 | 221. | 1.255814 | 318. | 1.069767 |
| 28. | 0.8828451 | 125. | 1.031381 | 222. | 1.069767 | 319. | 1.295349 |
| 29. | 0.9853556 | 126. | 1.062761 | 223. | 1.088372 | 320. | 1.018605 |
| 30. | 0.792887 | 127. | 1.152720 | 224. | 0.9279069 | 321. | 1.067442 |
| 31. | 0.8619246 | 128. | 1.184100 | 225. | 1.155814 | 322. | 1.062791 |
| 32. | 0.9811715 | 129. | 1.056485 | 226. | 1.093023 | 323. | 1.097674 |
| 33. | 0.956067 | 130. | 1.002092 | 227. | 0.9720929 | 324. | 1.104651 |
| 34. | 0.962343 | 131. | 1.014644 | 228. | 1.123256 | 325. | 1.076744 |
| 35. | 0.9100418 | 132. | 1.169456 | 229. | 1.041860 | 326. | 1.006977 |
| 36. | 0.956067 | 133. | 1.052301 | 230. | 0.9209302 | 327. | 1.369767 |
| 37. | 1.106694 | 134. | 0.8472803 | 231. | 0.8999999 | 328. | 0.9139535 |
| 38. | 0.9811715 | 135. | 1.033473 | 232. | 0.9069767 | 329. | 1.051163 |
| 39. | 1.087866 | 136. | 0.9456066 | 233. | 1.074419 | 330. | 1.039535 |
| 40. | 0.9079498 | 137. | 0.9832635 | 234. | 1.139535 | 331. | 1.034884 |
| 41. | 0.8995816 | 138. | 1.115063 | 235. | 1.051163 | 332. | 0.9860464 |
| 42. | 0.9309623 | 139. | 1.046025 | 236. | 0.8651162 | 333. | 1.055814 |
| 43. | 1.027197 | 140. | 1.303347 | 237. | 1.004651 | 334. | 1.032558 |
| 44. | 0.9895397 | 141. | 1.054393 | 238. | 0.8906976 | 335. | 1.158139 |
| 45. | 0.9916317 | 142. | 1.052301 | 239. | 0.9953489 | 336. | 1.074419 |
| 46. | 0.9414226 | 143. | 1.110879 | 240. | 1.079070 | 337. | 1.013953 |
| 47. | 0.9539748 | 144. | 0.9937238 | 241. | 0.9418605 | 338. | 0.9860464 |
| 48. | 1.018828 | 145. | 1.056485 | 242. | 0.9558139 | 339. | 0.9697674 |
| 49. | 0.9351463 | 146. | 0.9539748 | 243. | 1.018605 | 340. | 1.041860 |
| 50. | 0.9916317 | 147. | 1.004184 | 244. | 0.9860464 | 341. | 0.9581395 |
| 51. | 0.9539748 | 148. | 0.9853556 | 245. | 0.8930232 | 342. | 1.034884 |
| 52. | 0.8807531 | 149. | 0.9539748 | 246. | 0.844186 | 343. | 1.006977 |
| 53. | 0.9581589 | 150. | 0.9790794 | 247. | 0.8697674 | 344. | 0.9767441 |
| 54. | 1.039749 | 151. | 1.004184 | 248. | 0.8511628 | 345. | 0.9976743 |
| 55. | 0.9456066 | 152. | 0.9351463 | 249. | 0.9488372 | 346. | 0.9325582 |
| 56. | 1.006276 | 153. | 1.025105 | 250. | 1.002326 | 347. | 1.041860 |
| 57. | 1.083682 | 154. | 1.025105 | 251. | 0.9255813 | 348. | 1.025581 |
| 58. | 1.016736 | 155. | 0.9769874 | 252. | 0.8139535 | 349. | 0.9348837 |
| 59. | 1.023013 | 156. | 0.9665271 | 253. | 0.8162791 | 350. | 1.023256 |
| 60. | 1.127615 | 157. | 1.066946 | 254. | 0.988372 | 351. | 1.102325 |
| 61. | 0.9686192 | 158. | 1.138075 | 255. | 0.9279069 | 352. | 1.027907 |
| 62. | 1.050209 | 159. | 0.9895397 | 256. | 1.123256 | 353. | 0.7604651 |
| 63. | 0.9748953 | 160. | 0.9665271 | 257. | 1.125581 | 354. | 0.9418605 |
| 64. | 0.916318 | 161. | 0.960251 | 258. | 0.988372 | 355. | 1.016279 |
| 65. | 1.004184 | 162. | 1.048117 | 259. | 1.000000 | 356. | 0.9232558 |
| 66. | 0.8284519 | 163. | 1.077406 | 260. | 0.9372093 | 357. | 0.9395348 |
| 67. | 0.9539748 | 164. | 0.9874476 | 261. | 0.9093023 | 358. | 0.9697674 |
| 68. | 0.834728 | 165. | 0.9790794 | 262. | 0.9418605 | 359. | 1.006977 |
| 69. | 0.9644352 | 166. | 0.8640167 | 263. | 1.006977 | 360. | 0.9953489 |
| 70. | 1.073222 | 167. | 0.9539748 | 264. | 0.8860465 | 361. | 0.9813952 |
| 71. | 1.058577 | 168. | 1.006276 | 265. | 0.9139535 | 362. | 1.004651 |
| 72. | 0.9832635 | 169. | 0.9539748 | 266. | 0.9744186 | 363. | 0.8372092 |
| 73. | 0.916318 | 170. | 1.094142 | 267. | 0.8790697 | 364. | 0.9279069 |
| 74. | 0.9288703 | 171. | 1.004184 | 268. | 1.065116 | 365. | 0.9325582 |
| 75. | 0.8744769 | 172. | 0.9958159 | 269. | 1.023256 | 366. | 0.8744186 |
| 76. | 1.048117 | 173. | 0.9539748 | 270. | 1.004651 | 367. | 0.9418605 |
| 77. | 1.025105 | 174. | 1.232218 | 271. | 1.011628 | 368. | 0.9767441 |
| 78. | 0.9832635 | 175. | 0.9414226 | 272. | 1.102325 | 369. | 1.037209 |
| 79. | 0.8870292 | 176. | 0.9267781 | 273. | 1.093023 | 370. | 0.7418604 |
| 80. | 0.9832635 | 177. | 0.9267781 | 274. | 0.8558139 | 371. | 0.8790697 |
| 81. | 0.9937238 | 178. | 0.9100418 | 275. | 1.209302 | 372. | 0.8418604 |
| 82. | 1.066946 | 179. | 1.035565 | 276. | 1.102325 | 373. | 0.8860465 |
| 83. | 1.274059 | 180. | 0.9769874 | 277. | 1.106977 | 374. | 0.8023255 |
| 84. | 0.9728034 | 181. | 1.052301 | 278. | 1.162791 | 375. | 0.9860464 |
| 85. | 1.002092 | 182. | 0.9686192 | 279. | 0.9069767 | 376. | 0.944186 |
| 86. | 0.956067 | 183. | 0.9790794 | 280. | 1.055814 | 377. | 0.9232558 |
| 87. | 0.956067 | 184. | 1.194561 | 281. | 1.132558 | 378. | 0.944186 |
| 88. | 0.9979079 | 185. | 1.100418 | 282. | 1.072093 | 379. | 0.9465116 |
| 89. | 0.9309623 | 186. | 1.104602 | 283. | 1.109302 | 380. | 0.8279069 |
| 90. | 1.012552 | 187. | 0.9958159 | 284. | 1.030232 | 381. | 0.9395348 |
| 91. | 1.000000 | 188. | 1.066946 | 285. | 1.060465 | 382. | 0.9697674 |
| 92. | 0.9497907 | 189. | 1.079498 | 286. | 1.018605 | 383. | 0.8604651 |
| 93. | 0.9895397 | 190. | 0.9686192 | 287. | 1.065116 | 384. | 0.8465116 |
| 94. | 0.960251 | 191. | 0.9267781 | 288. | 1.218605 | 385. | 0.9069767 |
| 95. | 1.000000 | 192. | 0.9351463 | 289. | 1.025581 | | |
| 96. | 1.033473 | 193. | 0.8451883 | 290. | 1.076744 | | |
| 97. | 0.960251 | 194. | 0.9769874 | 291. | 1.086046 | | |

According to FIG. 1, in 377 cases (97.9%) the obtained results were within span, which in the case of normal distribution should include 99% of results (area between 2 arrows on the FIGURE). The results were out of this span in 8 cases (12.1%).

Cows, whose milk oxygen levels were different from the established threshold (mean cO₂ value±3σ), were taken under special observation. From this group, 50% of cows were diagnosed clinical mastitis during the observation period.

The concentration of dissolved oxygen was measured with Clark-type oxygen sensor also in the milk from different tits of the infected with mastitis cows, milked manually. In milk probes of 12 ml the oxygen concentration was measured right after milking within 60 seconds. The results of the measurements of oxygen in milk from infected udder quarters of mastitic animals were notably different from the results in milk from healthy animals. In most probes of the milk from infected udder quarters of mastitic animals, the oxygen concentration was considerably lower in comparison with milk of healthy animals (approximately 2 times lower), milked in similar conditions. There were also probes from infected udder quarters, in which oxygen concentration was considerably higher than in milk from healthy animals, taken in similar conditions. In conclusion the measured oxygen concentrations in all milk probes from infected udder quarters of mastitic animals were drastically different from the mean value of oxygen concentration in milk from healthy animals.

The above-described procedure of measuring oxygen concentration in milk was also used for the determination of milk quality. In case, the measured oxygen concentration in milk probes from infected udder quarters was drastically different from the mean value of oxygen concentration in milk from healthy animals (difference more than 3 σ values), substandard milk was identified and this milk was separated on-line before reaching the milk tubes and directed to waste.

The mastitis sensor consisted of an oxygen sensor, a device for the digitalization of the sensor analogue output signal, an automatic data acquisition and processing system and a mastitis indicator, where the results were compared with the normalized mean value of oxygen concentration and in case of establishing significant difference (over 3 σ) in the results, a signal lamp lightened on the panel of the indicator. The mastitis sensor enables to generate a signal, which starts the system of on-line separation of substandard milk from quality milk if necessary. The mastitis sensor is placed in milking tubes or in small collecting tanks in milking system, calibrated according to the temperature of the testing place and the concentration of dissolved oxygen in milk is measured in real time.

In case the measured oxygen concentration in milk is considerably different from the mean value of oxygen concentration (normally the concentration of oxygen in milk is 65 to 75% of the oxygen saturation concentration at 38.6° C. or 4.30 to 4.95 mg/l accordingly; the oxygen saturation concentration at 38.6° C. is 6.60 mg/l) and the difference with the mean value is more than 3 σ values, the animal is likely to have subclinical or clinical mastitis; in case the difference is 2-3 σ values, additional examination of the animal is recommended and in case the difference is smaller, the animal is healthy. The oxygen concentration in milk from infected udder quarters of animals suffering from mastitis, is 2-3 times lower than normal (23-49% of oxygen saturation concentration at 38.6° C.) or on the other extreme equals to the oxygen saturation concentration (100%).

Results, obtained with the mastitis sensor, are displayed on the screen of the device in the form of a continuous or discrete colour scale (e.g. difference over 3 σ generates a red, difference between 2 to 3 σ generates a yellow and difference under 2 σ values generates a green indicator colour) or as a numerical output.

1. A method for the detection of mastitis in animals, comprising the following steps:
a) an animal is milked and the concentration of dissolved molecular oxygen in milk is measured non-invasively;
b) the concentration of dissolved molecular oxygen in milk is compared with typical concentration of dissolved molecular oxygen found in uninfected milk; and
c) in case the oxygen concentration in milk is significantly different from the typical oxygen concentration in uninfected milk (difference is more than 3 standard deviation σ values from the mean value of the typical concentration or other given threshold), mastitis is detected in the animal in real time course.

2. The method according to claim 1, wherein the concentration of dissolved oxygen in milk is measured with a fiberoptic, amperometric or potentiometric device.

3. The method according to claim 1, wherein an animal is milked with a vacuum milking system and the concentration of dissolved molecular oxygen in milk is determined in the milking system with no access of external air.

4. The method according to claim 1, wherein an animal is milked manually and the concentration of dissolved molecular oxygen in milk is determined not later than 60 seconds after the beginning of milking.

5. The method according to claim 1, wherein the concentration of dissolved molecular oxygen in milk is determined in one or more udder quarters.

6. The method according to claim 1, wherein the typical concentration of dissolved molecular oxygen in milk is determined as the mean dissolved oxygen concentration of the farm or the herd, or as the mean of the measured dissolved oxygen concentrations of an animal and the allowed deviation from the typical value of the concentration of the dissolved molecular oxygen is established on the basis of the mean value of the dissolved molecular oxygen of the farm or herd.

7. A method for the determination of milk quality in real time, wherein the milk of the animal, in whose organism mastitis has been detected according to claim 1, is determined as substandard.

8. The method according to claim 1, wherein the animal is a cow, a goat or a sheep.

9. Mastitis sensor for the application of the method according to claim 1, comprising a fiberoptic, amperometric or potentiometric device for the determination of oxygen concentration, a device for data acquisition and processing, mastitis indicator and a device, generating a signal for the automatic on-line elimination of substandard milk.
